(19) United States
(12) Patent Application Publication
(10) Pub. No.: US 2015/0196434 A1
(43) Pub. Date: Jul. 16, 2015

(54) Stretch Cloth Diaper Flat

(71) Applicant: Jenny Alaine Reel, Wake Forest, NC (US)

(72) Inventor: Jenny Alaine Reel, Wake Forest, NC (US)

(21) Appl. No.: 14/665,637

(22) Filed: Mar. 23, 2015

Related U.S. Application Data
(60) Provisional application No. 61/969,863, filed on Mar. 25, 2014.

(51) Int. Cl. A61F 13/531; A61F 13/49

(52) U.S. Cl. CPC A61F 13/531; A61F 13/49003; A61F 2013/530379

(57) ABSTRACT

A cloth diaper flat's fabric is formed from a blend of multiple different types of fibers, including spandex fibers. The spandex fibers comprise 10% or less of the fibers in the blend but extend throughout the entirety of the fabric's dimensions so as to extend throughout the entirety of the cloth diaper flat's dimensions. In at least some embodiments, the blend also includes at least 65% viscose from bamboo fibers and between 10% and 35% cotton fibers

### RELATED APPLICATIONS

The present application claims the benefit of U.S. Provisional Application 61/969,863, filed Mar. 25, 2014, the disclosure of which is incorporated herein by reference in its entirety.

### TECHNICAL FIELD

The present invention generally relates to a diaper, and particularly relates to a diaper flat.

### BACKGROUND

Cloth diaper flats are the world's oldest cloth diaper style. These simple, single-layered pieces of fabric have been used and loved as diapers for thousands of years because they can be folded to fit babies of many different shapes and sizes. Since they are typically made with just one layer of fabric, they are quick and easy to clean, and fast to dry.

In the last 30 years, the United States' cloth diaper market has changed such that more complex cloth diaper styles (such as “all-in-one cloth diapers” and “fitted diapers”) are the most popular choices on the market. Unfortunately, even the best of these more complex styles cannot consistently give the customized fit that a cloth diaper flat can offer; an “all-in-one” diaper that fits beautifully on a 3-month-old child may fit poorly on that same child two months later, whereas the simple, single-layer diaper flat can be folded for a perfect fit on that child even as their shape changes.

In spite of the benefits mentioned so far for flat diapers (washability, fast-drying, and customization), their popularity waned with the advent of the more complex diaper styles because of the difficulties of folding flat diapers into the appropriate shapes and then fastening the diapers around the child in such a way that the diapers would remain secure and no urine or feces would leak. Most flat diapers were also not absorbent enough to be effective by themselves, and techniques for increasing absorbency (such as using two diapers at once, or adding extra inserts) created added bulk that made it difficult for children to fit into standard off-the-rack clothing.

The advent of “luxury flats” in the last 15 years has attempted to deal with some of the absorbency and fit issues that were plaguing otherwise-useful flats. Made of absorbent component fibers like viscose from bamboo (rayon) or hemp and cotton, they were sometimes made with double-sided terry fabric that had a small amount of natural stretch along the lengthwise grain of the fabric due to the fabric knit. While these diapers were a step forward, they are quite bulky and they still do not solve the fit issues that plague caregivers, as the diaper can only stretch slightly and in only one direction. Like an article of clothing such as a sweater, these luxury flat diapers made from knit fabrics have some ability to stretch, but only some and in one direction. Clothing designers create sweaters so that they stretch across the width of the body to accommodate the wearer's movements, particularly when pulling the sweater on and off.

Since flat diapers will be folded in various complex ways before being worn, however, the stretch that characterizes the flat in a single layer is no longer as beneficial in the folded diaper flat. In the folded diaper flat, the stretchiest part of the fabric (depending on the particular diaper fold and fabric orientation that is chosen) will not always be positioned where it can ease the process of getting a secure fit with the diaper. Further when multiple layers of the knit fabric are stacked together (as they are with a folded flat that is ready to be fastened on a child) the resulting diaper will only stretch as much across the child's hips as the least stretchy grain of fabric will allow.

### SUMMARY

A cloth diaper flat herein is made from a fiber blend that includes spandex. For example, the spandex is infused or otherwise distributed throughout substantially the entire fabric of the diaper flat, as opposed to just along certain edges or seams. The spandex-infused fabric of the diaper flat advantageously stretches not just along the lengthwise and crosswise grains, nor even simply on the bias, but in every conceivable direction. The omni-dimensional stretchability of the spandex-infused diaper flat yields a diaper with a truly responsive fit, no matter which of the many possible flat diaper folds are used to create the diaper (e.g. Kite fold, Airplane fold, Neat fold, Gaynor's fold, Bikini twist etc.,) Accordingly, even when the diaper is folded into complex shapes, and even when the different layers of the folded diaper are oriented in various different directions, the spandex still allows for significant stretch in every direction.

### BRIEF DESCRIPTION OF THE DRAWINGS

FIGS. 1A-1D are top-down views of a cloth diaper flat according to various embodiments herein.

FIGS. 2A-2D are cross-sectional views of a cloth diaper flat along the cross cuts shown in FIG. 1, according to various embodiments herein.

FIG. 3 is a block diagram of a flat diaper folded in preparation for use, according to one or more embodiments.

### DETAILED DESCRIPTION

One or more embodiments herein include a cloth diaper flat. The cloth diaper flat's fabric is formed from a blend of multiple different types of fibers. For example, strands or threads of one material (called fibers) are woven, knitted, or otherwise blended together with strands or threads of one or more other materials. Notably, the blend herein includes spandex fibers. The spandex fibers comprise less than 10% of the fibers in the blend but extend throughout the entirety of the fabric's dimensions so as to extend throughout the entirety of the cloth diaper flat's dimensions.

By incorporating spandex into the diaper flat's fiber blend itself, as opposed to just lining the diaper flat's edges with spandex, the diaper flat has omni-dimensional stretchability. That is, using spandex in the diaper blend allows the diaper to stretch in every possible direction. The omni-dimensional stretchability of the diaper flat means that the flat can be formed into a diaper with a truly responsive fit, no matter which of the many possible flat diaper folds are used to create the diaper (e.g. Kite fold, Airplane fold, Neat fold, Gaynor's fold, Bikini twist etc.,). This therefore facilitates use of even multitudinous complex flat diaper folds.

Spandex also allows for increased comfort for active diaper-wearers. The stretchiness of the fabric makes it much easier to achieve and maintain a custom fit, preventing urine and feces from escaping the fastened diaper and diminishing diaper rash. Yet the resilience of the spandex fiber also allows the diaper to spring back into its original shape quickly after use, allowing for continued effectiveness of the product even with long-term use.

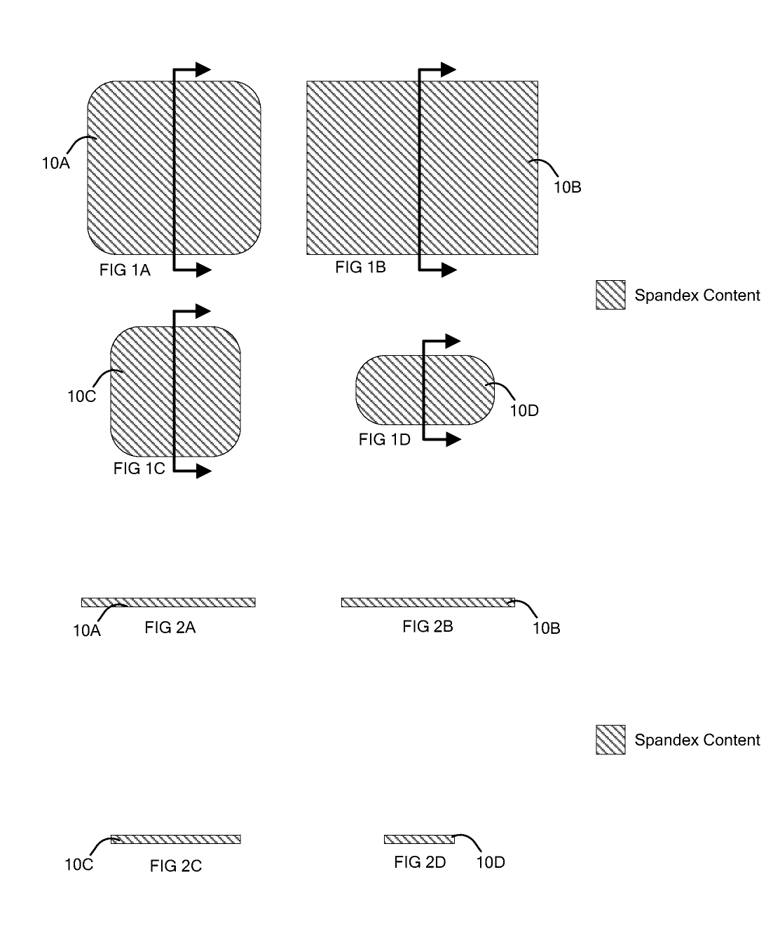

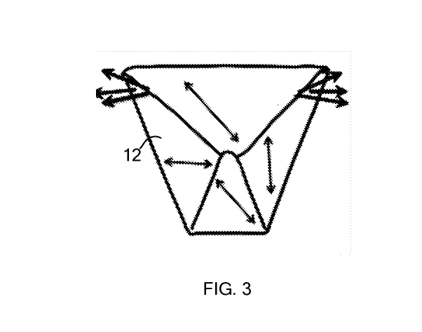

The accompanying figures illustrate a few exemplary diaper flats and diaper flat folds in this regard. FIGS. 1A-1D are top-down views of a cloth diaper flat 10 according to various embodiments. FIGS. 2A-2D are cross-sectional views of a cloth diaper flat 10 taken along the cross cuts respectively illustrated in FIGS. 1A-1D. Notably, the cloth diaper flat 10 has spandex infused or otherwise distributed substantially throughout the entire dimensions of that flat 10, since that spandex is incorporated into the fiber blend itself.

In more detail, FIGS. 1A and 2A show a spandex-infused square flat 10A with rounded edges that are configured for forming a serged/overlocked diaper. The square flat 10A has square edges in other embodiments despite not being shown. FIGS. 1B and 2B show a rectangular flat 10B. The rectangular flat 10B is configured for either being padfolded (folded into a rectangle and placed into a diaper cover, foregoing the use of diaper fasteners), or folded to into a square before a traditional diaper fold is performed. FIGS. 1C and 2C shows a smaller proportioned flat 10C configured to form a diaper for a newborn (those sizes are also used as additional absorbent inserts placed inside the diaper of an older child). FIGS. 1D and 2D show a rectangular flat 10D that has rounded edges and is dimensioned to be substantially twice as long as it is wide. In some embodiments, the flat 10D is configured to be padfolded (folded into a rectangle and placed in a diaper cover, foregoing the use of diaper fasteners). In other embodiments, the flat 10D is configured to be folded in half and fastened on smaller babies in a fashion similar to that of “pre-fold” cloth diapers.

FIG. 2 shows a diaper flat 12 that has been folded in preparation for use, using the popular folding technique called the “kite fold”. As shown by bidirectional arrows in FIG. 2, the diaper flat 12 advantageously stretches in multiple directions determined as being required for proper fit. FIG. 2 also illustrates orientations of the stretchiest grain of the fabric on each of the various layers of the folded flat. In at least some embodiments, the diaper will stretch well horizontally even in a folded diaper flat with its varying grain orientation.

In at least some embodiments, one or more of the illustrated flats 10 or 12 are substantially larger than a pre-fold cloth diaper.

Of course, a diaper flat herein can be cut into other shapes and/or sizes. 15″×15″ would be a size appropriate for newborn infants, while a 32″×32″ would fit an older toddler. Many possible sizes and shapes exist between these extremes. In the currently preferred embodiment, a diaper flat system herein includes diaper flats offered in multiple sizes: namely, 20″×20″, 23″×23″, 15″×30″, 27″×27″, and 30″×30″ sizes.

In at least some embodiments, the fabric has a weight and blend type configured to absorb moisture while at the same time minimizing bulk. Ultra absorbent component fibers like hemp and bamboo, for example, require fewer diaper inserts to be added, which makes for less bulky diapers and ultimately allows the wearer to fit more easily into standard store-bought clothes. Regardless, in some embodiments, the fabric's blend includes cotton, rayon, hemp, soy, or other moisture absorbing fibers and/or has a weight between 6 and 10 ounces per yard.

To maximize absorbency, the fabric in at least some embodiments has the highest possible percentage of viscose from bamboo. Viscose from bamboo is not only soft, it wicks moisture quickly, which prevents leaks, and it is twice as absorbent as cotton. In at least some embodiments, the blend alternatively or additionally incorporates organic cotton as it has a higher tensile strength and it adds the pleasant natural loft.

The weight of the diaper flat in some embodiments, at 6-10 ounces per square yeard, is more substantial than that of standard cotton flats, without being so thick that it would cause bulk. The flat in at least one embodiment also features a single-sided french-terry knit fabric that reduces bulk (compared to standard terry knits) while still offering a textured surface to aid grip with today's popular grip diaper fasteners. The smooth side of the fabric not only further reduces bulk (compared with double loop terry luxury flats), it provides a surface that is easy to penetrate with diaper pins. Users can choose whether they want the smooth side or the terry side facing out, and can choose any type of diaper fastener they prefer.

The thread used for stitching in some embodiments is a high quality, medium weight, Tex 27 or greater thread, durable enough to withstand rigorous cloth diaper laundering routines.

In at least some embodiments, spandex fibers comprise between 3% and 7% of the fibers in the blend, viscose from bamboo fibers comprise at least 65% of the fibers in the blend, and/or cotton fibers comprise between 10% and 32% of the fibers in the blend. In one particular embodiment, for example, spandex fibers comprise 5% of the fibers in the blend, viscose from bamboo fibers comprise 70% of the fibers in the blend, and cotton fibers comprise 25% of the fibers in the blend.

Alternatively or additionally, the fabric in some embodiments weighs between 6 and 10 ounces per square yard (e.g., 8 ounces per square yard).

Alternatively or additionally, the fabric in some embodiments is a single sided terry fabric, such as a single-side French terry fabric. The other side of the fabric in some embodiments is a smooth knit side.

Those skilled in the art will recognize that the present invention may be carried out in other ways than those specifically set forth herein without departing from essential characteristics of the invention. The present embodiments are thus to be considered in all respects as illustrative and not restrictive, and all changes coming within the meaning and equivalency range of the appended claims are intended to be embraced therein.

What is claimed is:

1. A cloth diaper flat whose fabric comprises a blend of multiple different types of fibers, including spandex fibers, wherein the spandex fibers comprise 10% or less of the fibers in said blend but extend throughout the entirety of the fabric's dimensions so as to extend throughout the entirety of the cloth diaper flat's dimensions.

2. The cloth diaper flat of claim 1, wherein the spandex fibers comprise between 3% and 7% of the fibers in said blend.

3. The cloth diaper flat of claim 1, wherein said blend also includes viscose from bamboo fibers.

4. The cloth diaper flat of claim 3, wherein the viscose from bamboo fibers comprise at least 60% of the fibers in said blend.

5. The cloth diaper flat of claim 3, wherein the viscose from bamboo fibers comprise at least 70% of the fibers in said blend.

6. The cloth diaper flat of claim 1, wherein said blend also includes one or more additional fibers selected from the group comprising cotton, hemp, soy, and lyocell fibers.

7. The cloth diaper flat of claim 1, wherein said blend also includes cotton fibers that comprise between 10% and 40% if the fibers in said blend.

8. The cloth diaper flat of claim 1, wherein the spandex fibers comprise between 3% and 7% of the fibers in said blend, wherein viscose from bamboo fibers comprise at least 65% of the fibers in said blend, and wherein cotton fibers comprise between 10% and 32% of the fibers in said blend.

9. The cloth diaper flat of claim 1, wherein the spandex fibers comprise 5% of the fibers in said blend, viscose from bamboo fibers comprise 70% of the fibers in said blend, and cotton fibers comprise 25% of the fibers in said blend.

10. The cloth diaper flat of claim 1, wherein the fabric weighs between 6 and 10 ounces per square yard.

11. The cloth diaper flat of claim 1, wherein the fabric weighs between 7 and 9 ounces per square yard.

12. The cloth diaper flat of claim 1, wherein the fabric weighs 8 ounces per square yard.

13. The cloth diaper flat of claim 1, wherein the fabric is a single-sided terry fabric.

14. The cloth diaper flat of claim 1, wherein the fabric is a single-sided French terry fabric.

15. The cloth diaper flat of claim 1, wherein the fabric has one smooth knit side and one French terry side.

16. The cloth diaper flat of claim 1:
wherein the spandex fibers comprise 5% of the fibers in said blend, viscose from bamboo fibers comprise 70% of the fibers in said blend, and cotton fibers comprise 25% of the fibers in said blend;
wherein the fabric weighs 8 ounces per square yard;
wherein the fabric is serged with a three or four thread stich using Tex 27 or greater polyester thread;
wherein the fabric has one smooth knit side and one French terry side.

17. A cloth diaper flat having spandex fibers blended throughout the diaper flat's fabric along with one or more other non-spandex fibers, wherein the cloth diaper flat has omni-directional stretchability.

18. The cloth diaper flat of claim 17, wherein the diaper flat's fabric weighs 8 ounces per square yard.

19. The cloth diaper flat of claim 17, wherein the spandex fibers comprise 10% or less of the diaper flat's fabric.

20. The cloth diaper flat of claim 17, wherein viscose from bamboo fibers comprise at least 65% of the diaper flat's fabric.

21. A cloth diaper flat with 1% to 10% spandex content.

22. The cloth diaper flat of claim 20, made from fabric that weighs 8 ounces per square yard.
